(19) United States
(12) Patent Application Publication
(10) Pub. No.: US 2006/0018965 A1
(43) Pub. Date: Jan. 26, 2006

(54) Solid oral dosage form containing seamless microcapsules

(76) Inventors: Joey Moodley, Athlone (IE); Ivan Coulter, Dublin (IE)

Correspondence Address: JACOBSON HOLMAN PLLC, 400 SEVENTH STREET N.W., SUITE 600, WASHINGTON, DC, 20004, US

(21) Appl. No.: 11/236,549

(22) Filed: Sep. 28, 2005

Related U.S. Application Data
(63) Continuation of application No. PCT/IE04/00049, filed on Mar. 29, 2004.

(51) Int. Cl. A61K 9/26

(52) U.S. Cl. 424/470

(57) ABSTRACT

This invention relates to a solid oral dosage form containing one or more pharmaceutically active ingredients solubilised or suspended in a pharmaceutically acceptable solvent or liquid phase and encapsulated in seamless controlled release microcapsules. Accordingly the pharmaceutically acceptable solvent or liquid phase may range from aqueous phase, organic solvent(s), glycols, oils and derivatives of including mono-,di, and triglycerides of short, medium and long chain fatty acids. The microcapsules have a diameter of <1 mm to 8 mm and a drug loading of up to 90%. Additionally the microcapsules may be coated to release the pharmaceutically active ingredient at specific sites and for predetermined rates.

### FIELD OF THE INVENTION

The present invention relates to a solid oral dosage form comprising a multiplicity of seamless microcapsules containing a pharmaceutically active ingredient solubilised or dispersed in a pharmaceutically acceptable solvent or liquid phase.

### BACKGROUND

Drugs for use in therapy and prophylaxis of various medical conditions have varying solubility characteristics ranging from insoluble to lipid soluble and water soluble with varying pH sensitivities. This variation in solubility can affect the therapeutic effectiveness of drugs. Drug dissolution is a prerequisite to drug absorption. Except in very special cases, drugs cannot be absorbed until they are solubilised. If a drug is already in solution at the time of administration, its absorption across the gastrointestinal tract and hence its bioavailability is rapid resulting in a fast therapeutic effect. Rapid or instantaneous bioavailability is characterised by significant blood levels within about 10 to 60 minutes after administration of the drug.

Drugs that are poorly water soluble and/or sensitive to pH must be formulated in a way that improves their solubility and hence their bioavailability. In general, a drug that is in solution or suspension when administered by the oral mute is rapidly, and frequently instantaneously, absorbed from the gastrointestinal tract resulting in a fast therapeutic action. However, in many cases, it is desirable to control the rate of drug absorption following oral administration in order to achieve the desired plasma profile or prolongation of action of the drug.

Numerous processes and formulations exist to enhance the solubility of poorly soluble drug compounds. Solubilisation of such drugs in solvents, oils, emulsions and microemulsions are well known to those skilled in the art and have been used to deliver such drugs orally. Such formulations are then encapsulated in soft gelatine capsules for oral administration. Soft gelatine encapsulation is a specialised process, whereby the end products, soft gelatine capsules do not lend themselves easily to further processing such as the addition of delayed or sustained release coatings. Therefore such dosage forms are particularly suited for instantaneous delivery of the encapsulated active drugs.

Particle size reduction processes such as milling, micronisation and spray drying are also well known to increase the surface area and hence solubilisation rates of poorly soluble drugs. U.S. Pat. No. 5,510,118 and U.S. Pat. No. 5,145,684 teaches the milling of such drugs in pharmaceutically acceptable solvents to produce nanosuspensions of the drugs with the resulting enhanced solubility of the drug. The production of nanosuspension/nanodispersion of poorly soluble drugs can be performed by using a double nozzle spray drying process. These nanosuspensions or nanodispersions result in increased dissolution rates of the active and increased absorption and bioavailability. However for administration as a solid oral dosage form with controlled and/or sustained release characteristics, the nanodispersions or nanosuspensions are subsequently dried to produce free flowing powder for further processing into a solid oral dosage form such as tablets, minitablets which can then be coated with a controlled or sustained release polymers to control the delivery rate of the active. Recovery of the nanoparticle size distribution on dissolution of these dosage forms is however limited often resulting in reduced dissolution rate of the active.

There is a need therefore for an oral formulation or process which can be used to administer the solubilised and/or dispersions including nanosuspensions of the active ingredients in a manner which allows the formulation to be subsequently coated to deliver the active at a predetermined site of absorption and/or at a predetermined rate of delivery consistent with the optimum absorption and bioavailability or plasma profile of the drug.

Additionally there is a need for an oral formulation which can be used to administer one or more active ingredients of differing solubility which are released in a predetermined manner to target sites in the gastrointestinal tract so as to achieve maximum absorption at the site of release of active ingredient.

There is also a need for an oral formulation which allows for release of an active ingredient in the gastrointestinal tract in a manner which minimizes high local concentrations of solid active ingredient. Multiparticulate drug delivery systems by their nature allow the release of the active ingredient over a larger surface area of the gastrointestinal tract thereby minimising high localised drug concentration for drugs which are irritants to the gastrointestinal tract

Drugs which are poorly permeable are often administered with one or more permeability enhancers to enhance their permeability and absorption. Numerous potential absorption enhancers have been identified Medium chain fatty acids and triglycerides have demonstrated the ability to enhance the absorption of hydrophilic drugs across the gastrointestinal mucosa (Pharm. Res., 1994, 11, 1148-54), (Pharm. Res. 1993, 10, 857-864). U.S. Pat. No. 4,656,161 (BASF AG) discloses a process for increasing the bioavailability of heparins and heparinoids by adding non-ionic surfactants with a fatty acid, a fatty alcohol, an alkylphenol or a sorbitan or glycerol fatty acid ester. It is also known that the administration of medium chain fatty acids and derivatives of, including amino acid derivatives in combination with the drug can enhance permeability of the drug.

For maximum enhancement, it is desirable that both enhancer and drug are in solution at the same rate. The solubilities of the enhancer and drug are often different resulting in different rates of solubilisation of the enhancer and drug and hence loss of bioavailability of the drug in comparison with bioavailability from a solution of the drug and enhancer. In addition poorly permeable drugs including macromolecular drugs and biotechnology drugs such as peptides, proteins, vaccines, oligosaccharides, polysaccharides including TRH, Calcitonin, Leoprolide acetate, alendronate, vasopressin, desmopressin and antisense oligonucleotides are acid labile and cannot be delivered to the stomach as a solution.

The use of essential oils to enhance bioavailability by reducing cytochrome P450 metabolism and/or P-glycoprotein regulated transport of the active out of the blood stream back into the gut is also known, (U.S. Pat. No. 5,66,386 to AvMax Inc. and others).

There is a need therefore for a controlled release technology which will allow the delivery of a drug and enhancer in solution to the optimum site of absorption/action in the gastrointestinal tract.

### SUMMARY OF THE INVENTION

The invention provides a controlled release formulation in solid unit dosage form, said formulation comprising a multiplicity of seamless microcapsules, each of which microcapsule contains one or more active ingredient in a liquid phase and having a predetermined release rate of active ingredient in the gastrointestinal tract following administration. The microcapsules collectively have one or more rates of release of active ingredient dependent on a predetermined permeability of the respective microcapsules. The microcapsules can be administered as a sprinkle on food or filled into a hard gelatine capsule or tabletted as a matrix, bilayer or multiple layer tablets. The final dosage form can be further coated to release the microcapsules at predetermined rates and/or site of the gastrointestinal tract.

The microcapsules of the formulation according to the invention effectively allow one to administer solutions or suspensions of active ingredients as if they were multi-particulate solid oral dosage forms. The microcapsules release their contents to the gastrointestinal tract in a manner which minimises high local concentrations of active ingredient which might otherwise result in irritation and other undesirable side effects, but additionally the drug is released in an already solubilised form which aids absorption.

The individual microcapsules suitably have an average diameter in the range 100-10,000 μm, more particularly in the range 250-8,000 μm and especially in the range 500-5,000 μm.

The walls of the microcapsules will suitably have a thickness in the range 30-1,000 μm, especially in 100-500 μm.

The walls of the microcapsules have a predetermined permeability by which is meant they either dissolve or have a natural permeability to gastrointestinal fluid so that active ingredient is released therefrom as desired in the gastrointestinal tract.

Microcapsules with naturally permeable walls can be soluble, porous or, alternatively, the solubility or porosity can develop as a result of the change in environmental conditions as the formulation passes through the gastrointestinal tract.

Thus, the wall of each microcapsule can be formed of a pharmaceutically acceptable, film forming polymer or mixture of polymers which is soluble in the gastrointestinal tract.

In a preferred embodiment, the wall of the microcapsule is formed of soft or hard gelatine (such as a bovine or porcine gelatine material) or other soft gel materials made from suitable polymers. Examples of other soft gel materials include starches that form a soft gel or high molecular weight polyethylene glycols or Agar/Agar. However, in practice any material that can dissolve in the gastrointestinal fluid can be used. Such a material can be a material which is incorporated in the wall and which dissolves in the gastrointestinal fluid, namely a porosigen.

The wall of the microcapsule will have a polymeric composition and/or structure which allows for fast release and thus fast absorption of active ingredient once the wall is partially or wholly permeable.

Once the wall of the microcapsule has the requisite permeability, the active ingredient in its liquid carrier passes into the gastrointestinal tract for immediate absorption. The liquid carrier can include one or more auxiliary agents with bioavailability and/or cytoprotective, especially gastroprotective, enhancing properties.

The formulation according to the invention can comprise a blend of microcapsules having walls of variable, but predetermined permeability so as to achieve immediate, intermediate or sustained release of active ingredient over a given time period in the gastrointestinal tract.

Accordingly, it will be appreciated that the formulation according to the invention has advantages over conventional solid microparticles in which the release of active ingredient is dependent on progressive solubilisation in the gastrointestinal tract.

The permeability of the walls of the microcapsules can be dependent on pH, temperature and other physical conditions prevailing within the gastrointestinal tract.

The controlled release properties of the microcapsules according to the invention will principally be a function of the thickness of the walls of the microcapsules, or by including pH dependent substances such as polymers or shellac.

The microcapsules can contain two or more active ingredients having different solubilities in the aqueous environment of the gastrointestinal tract, but with compatible solubility or suspending capability in the liquid medium of the microcapsule. Alternatively, the microcapsules can contain single active ingredients solubilised in different media but which can be released for simultaneous absorption from microcapsules having walls of different materials but with similar or different permeability characteristics.

The microcapsules can also contain two or more active ingredients having different half lives following absorption from the gastrointestinal tract

The solubility of the or each active ingredient can be dependent on the pH of a given region of the gastrointestinal tract

The microcapsules can be manufactured so that they release their contents in the gastrointestinal tract at a point at which the drug is most soluble. This feature enables one to maximise absorption because the microcapsules release their contents when the pH conditions are optimal.

The pH internally of the microcapsules can be optimised by the use of an acid or an alkaline solution, as required to maximise the absorption of active ingredients that are pH sensitive.

An example of a formulation with microcapsules capable of achieving immediate, intermediate and sustained release of active ingredient over a given time period in the gastrointestinal tract might be a formulation for use in the treatment of the common cold and influenza Such formulations are conventionally multiple active ingredient formulations. The common cold and influenza are each characterised by a variety of symptoms which cannot generally be alleviated by a single active ingredient For example, it might be desired to administer an antihistamine, a decongestant and one or more cough suppressants.

It may also be desirable to add a diuretic such as hydrochlorothiazide and/or an anti hypertensive such as Losartan.

The formulation according to the present invention is ideally suited for use as a multiple active ingredient formulation.

The microcapsules can contain an active ingredient suspended or solubilised in a solution of a permeability enhancer in a ratio of enhancer to drug ranging from 0 drug: 100 enhancer to 100 drug: 0 enhancer which can be released for simultaneous absorption from microcapsules having walls of different materials but with similar or different permeability characteristics.

Additionally microcapsules containing the enhancer solution without the drug can be released at a slower rate from microcapsules containing the drug and enhancer solutions to maximise the permeably enhancement of the unabsorbed drug in the gastrointestinal track.

The walls of the microcapsules can have inherent mucoadhesive properties and thus bind to the wall of the gastrointestinal tract during release of active ingredient therefrom.

The walls of the microcapsules can also have inherent enteric coating properties.

Some or all of the microcapsules in the formulation can have an enteric coating, for example, an outer membrane of shellac or other enteric coating.

It will be appreciated that one can achieve selective absorption of active ingredients using a formulation in accordance with the invention.

Essentially the solution in which the drug is dissolved or suspended in any pharmaceutically acceptable solvent or liquid phase provided the solvent or liquid phase does not dissolve the wall of microcapsules.

The liquid phase can suitably be an oil. When the oil is soya bean oil or mineral oil, the active agent would typically form a suspension in such oils. The liquid phase can also be an aqueous phase. The oil phase can be an oil of nutritional benefit or having pharmacological activity such as omega-3-fatty acids and omega-6 fatty acids

Such aqueous phases include, for example, high molecular weight liquid polyethylene glycols and short or medium chain mono-, di and/or tri-glycerides.

Suitably the or each active ingredient can be dissolved in a polyol in the core of the microcapsules. Examples of polyols are polyethylene glycols and cellulose derivatives.

The core can also contain one or more auxiliary agents selected from a pH controlling agent, an anti-oxidant, a humectant, a surfactant and a vasodilator.

Suitable pH controlling agents include, for example, citric acid, fumaric acid, sodium citrate and the like.

Examples of anti-oxidants are sodium metabisulphite, butylated hydroxyanisole and butylated hydroxytoluene or a mixture thereof.

Examples of suitable humectants are glycerol and sorbitol.

Examples of suitable surfactants include sodium lauryl sulphate, diethylene glycol monostearate, propylene glycol monostearate, polyethylene glycol, polysorbates and polyvinyl alcohol or mixtures thereof.

The microcapsules can contain up to 90% by weight of active ingredient. However, in general the microcapsules will contain between 25 and 75% by weight of active ingredient

Each formulation will suitably contain between 10 and 300 microcapsules, preferably between 100 and 250 microcapsules.

The microcapsules will suitably be administered by loading them into a hard gelatine capsule which will be swallowed in the normal way or by loading them into another container such as a sachet, the contents of which can then be swallowed or sprinkled onto food and swallowed. In certain special circumstances the microcapsules may be incorporated into a tablet in a way which maintains their integrity.

A formulation in accordance with the invention can be designed so as to achieve fast medium and slow release of one or more active ingredients. Thus, it will be appreciated that the formulation in accordance with the invention can be used to achieve maximum bioavailability resulting from maximum absorption of one or more active ingredients from the gastrointestinal tract

The formulation according to the invention is suitable for the administration of a wide range of active ingredients.

For example the formulation can be used in the case of insoluble active ingredients such as nifedipine, lipid soluble active ingredients such as gemfibrizol, and pH sensitive active ingredients such as captopril.

The formulation according to the invention is also suitable for the administration of active ingredients which are sensitive to the pH environment in the stomach, such as omeprazole and other proton pump inhibitors used in anti-ulcer treatment.

The formulation according to the invention can also be used to improve the bioavailability of active ingredients such as terfenadine which have a low oral bioavailability.

The formulation according to the invention can also be used to dramatically increase the absorption of active ingredients which are poorly absorbed from or are destroyed in the gastrointestinal tract such as captopril, cyclosporin, calcitonin, heparins and heparinoids.

Suitable classes of therapeutic agents which can be delivered using this invention include but are not limited to poorly water soluble drugs such as cardiovascular agents, lipid lowering agents, anti-diabetic agents, anti-epileptics, anti-infectives, anti-fungal agents, anti-viral agents, antipsychotic agents, immunosuppressants, protease inhibitors, cyclic peptides.

Suitable classes of therapeutic agents which can be delivered using this invention include but are not limited to peptides, proteins, vaccines, and oligonucleotides, including non-covalent or covalent modified versions thereof.

An example of a formulation of an insoluble drug providing both immediate and sustained release in accordance with the invention comprises a capsule containing a mixture of immediate and sustained release seamless microcapsules of varying sizes, for example. 1-8 mm. The active ingredient, for example, nifedipine or cyclosporine is dissolved or suspended, as appropriate, for example, in a polyethylene glycol, an oil-base, a suspension of a polyethylene glycol and a mineral oil, or polyoxethylene sorbitan fatty acid esters or a suitable combination of an oil and surfactants or an emulsion or microemulsion preconcentrate. An outer membrane liquid for example, gelatin and an inner active ingredient liquid are combined to form droplets i.e., microcapsules. The microcapsules are passed through a cooling system, for example, oil. The seamless microcapsules containing the active ingredient are removed from the cooling system and cleaned and dried in separate facilities.

The sustained action of the microcapsules is determined by the thickness of the outer membrane or by the addition of a variety of sustained release polymers to the inner liquid or the outer membrane liquid, for example, polymethacrylates, cellulose derivatives, polyvinylpyrrolidones. As indicated above, the microcapsules can be formulated to release the active liquid ingredient at a specific absorption site, for example by the use of pH dependent polymethacrylates.

The invention provides an oral formulation which allows for protection of the active ingredient from harsh environments such as gastric acid and intestinal proteases and other degradative processes. Enteric coating protects drugs from release into acidic environments, protease and nuclease inhibitors reduce proteolytic and nucleic acid degradation while mucoadhesive coatings minimise exposure to degradative enzymes,

For maximum protection from degradative processes, it may be desirable that the drug to be protected is formulated as a solution or emulsion that contains the appropriate degradation inhibitor, such as a nuclease or protease inhibitor. Otherwise, it may be necessary to formulate the drug and degradative process inhibitor as separate minicapsules. In addition, it may be necessary to coat the minicapsules with both an outer enteric coat and an inner mucoadhesive coating to reduce the exposure time to degradative enzymes thereby mitigating degradation.

The invention will be further illustrated by the following examples:

General Outline of Seamless Microcapsule Formation

The principle of seamless microcapsule formation is the utilisation of “surface tension,” when two different solutions (which are not or hardly dissolved with each other) contact each other, which works by reducing the contact area of the two different solutions.

After encapsulating the core solution, which is ejected through an orifice with a certain diameter, with the shell solution which is also ejected through an outer orifice, the encapsulated sphere is then ejected into a cooling or hardening solution and the outer shell solution is gelled or solidified. Thus seamless microcapsules are formed.

The core solution is mainly a hydrophobic solution or suspension. The outer shell solution is normally gelatin based. However, a hydrophilic solution can also be encapsulated with the existence of an intermediate solution, which can avoid the direct contact of the hydrophilic core solution with the outer shell.

With the nozzle having a single orifice, a capsule or a bead of shell/core mixed suspension can be processed. With the nozzle having two orifices (center and outer), a hydrophobic solution can be encapsulated. With the nozzle having three or more orifices seamless microcapsules for various applications can be processed.

### EXAMPLE 1

Nifedipine is dissolved in soybean oil and formed into seamless microcapsules according to the methods described in U.S. Pat. Nos. 5,478,508 and 5,882,680 with an outer gelatin coating as hereinbefore described. These microcapsules have a size distribution of 1-3 mm

The nifedipine microcapsules are then coated in a conventional manner with a cellulose polymer coating to provide a controlled release dissolution rate.

The coated microcapsules are finally encapsulated into a hard gelatin capsule shell.

The details for this example are as follows:

| | Weight % |
|---|---|
| Core Solution | |
| Nifedipine USP | 9.1 |
| Polyethylene Glycol (Grade 200; 300; 400; 600) | 90.9 |
| Intermediate Solution | |
| Vegetable Oil | 100.0 |
| Film Solution | |
| Gelatin | 12.0-22.5 |
| Sorbitol | 1.5-2.5 |
| Purified Water | 75-85 |
| Microcapsule Formation | |
| Ingredients | |
| Core Solution | 30-70 |
| Intermediate Solution | 5-25 |
| Film Solution | 15-65 |

The core solution is formed into seamless microcapsules with an intermediate solution layer and an outer hard gelatin film layer. The intermediate layer acts as a barrier preventing the core solution from migrating into the outer gelatin layer.

The microcapsules produced have a particle size range of between 1.00-3.00 mm.

Nifedipine Mean Dissolution Rate (1.25% sodiumlaurylsulphate)

| Time | Mg/Release | % Release |
|---|---|---|
| 5 min | 93.2 | 28.6 |
| 10 min | 190.3 | 58.4 |
| 15 min | 230.5 | 70.8 |
| 30 min | 279.1 | 85.7 |
| 45 min | 324.8 | 99.8 |

### EXAMPLE 2

Nifedipine seamless microcapsules are formulated as described in Example 1, however, the inherent release characteristics of the pellets are varied from Example 1.

These microcapsules provide a longer controlled release action

The details for this example are as follows:

| Ingredients | |
|---|---|
| Nifedipine Microcapsules (1.50-1.80 mm) | 500 Grams |
| Ammino Methacrylate Copolomer (Eudragit RL) | 5-50 w/w |
| Ammino Methacrylate Copolomer (Eudragit RS) | 50-95 w/w |
| Isopropyl Alcohol* | — |
| Acetone* | — |

*Used in processing, occurring in trace amounts in finished product

Place 500 grams of Nifedipine microcapsules prepared in the same way as Example 1 in a suitable fluidised bed system (eg Glatt GPCG 1 or Vector FL-M-1 Unit). Spray coat the microcapsules with a 6.25 solution comprising Eudragit RL (10% w/w) and Eudragit RS (90% w/w) dissolved in isopropyl alcohol/acetone. Talc was added simultaneously via an auger feeder to prevent agglomeration.

The resulting product consisted of a microcapsule coated with a rate-controlling polymer that provides a sustained release of Nifedipine throughout the gastrointestinal tract over a 12 or 24 hour duration The coated microcapsules are then filled into hard gelatin capsules.

### EXAMPLE 2A

Nifedipine seamless microcapsules were prepared in the same way as for the microcapsules in Example 1, however the inherent release characteristics of the microcapsules are varied from Example 1, by increasing the wall thickness of the outer gelatin layer of the microcapsule.

The microcapsules were placed in a suitable fluidised bed coater and spray coated with the same polymer coating formulation as in Example 2, thus providing a longer sustained release action typically 24 hours.

### EXAMPLE 3

Gemfibrozil (a liquid soluble drug) is formulated along with various surfactants and gelatin into seamless microcapsules of varying thickness.

A portion of these pellets are coated with a methacrylate polymer system and combined in a ratio of 4:1 with uncoated microcapsules, then filled into hard gelatin capsule shells, thereby providing a drug formulation having both immediate and sustained release dissolution characteristics.

### EXAMPLE 4

Captopril is dissolved in soy bean oil, and formed into seamless microcapsules with an outer gelatin coating. These microcapsules have a size distribution of 1-3 mm.

The microcapsules are then coated in a conventional manner with a cellulose polymer coating to provide a controlled release dissolution rate.

The coated microcapsules are finally encapsulated into a hard gelatin capsule shell.

### EXAMPLE 4A

| Core Solution | |
|---|---|
| Captopril | 60 grams |
| Polyethylene Glycol (grade 200; 300; 400; 600) | 100 grams |
| Citric Acid Anhydrous | pH Adjuster |
| Purified Water | 100 grams |

Intermediate Solution

As in Example 1

Film Solution

As in Example 1

Place 100 grams of Polyethylene Glycol (grade 200; 300; 400; 600) and 100 grams Purified Water in a suitable container and mix using a mechanical mixer. Add a suitable quantity of Citric Acid to the Glycol/Water mix, to bring to predetermined pH value. Add the Captopril to the solution. Additional Citric Acid maybe added if the predetermined pH value is not achieved.

The Theoretical Target Potency of the Core Solution should be in the range of 200-300 mg/g. The seamless microcapsules are produced as described in Examples 1 and 2.

### EXAMPLE 5

Captopril seamless microcapsules are formulated as described in Example 4, however, the inherent release characteristics of the microcapsules is varied from Example 4, by increasing the wall thickness of the microcapsules.

These microcapsules, when coated with the same polymer coating as in Example 4, provide a longer controlled release action.

### EXAMPLE 6

Cyclosporine is solubilised in a suitable medium chain triglyceride and formed into seamless microcapsules with an outer gelatin coating as herein before described. These microcapsules have a size distribution of 3-6 mm. The cyclosporine microcapsules are then encapsulated into a hard gelatin capsule shell.

### EXAMPLE 7

Cyclosporine dispersion is prepared and formed into seamless microcapsules with an outer gelatin coating as herein before described These microcapsules have a size distribution of 3-6 mm.

The cyclosporine microcapsules are then encapsulated into a hard gelatin capsule shelf.

### EXAMPLE 8

Cyclosporine is solubilised in a suitable medium chain triglyceride, and formed into seamless microcapsules with an outer gelatin coating as herein before described. These microcapsules have a size distribution of 3-6 mm.

Omega-3 fatty acids, are encapsulated into seamless microcapsules as described in Example 1. The cyclosporine microcapsules and omega-3 fatty acid microcapsules are mixed in the ratio of 100:300 and filled into hard gelatine capsules to give a cyclosporine content of 25 mg/capsule.

The cyclosporine microcapsules are then encapsulated into a hard gelatin capsule shell.

### EXAMPLE 8A

Cyclosporine is solubilised in a suitable medium chain triglyceride and formed into seamless microcapsules with a outer gelatin layer as described in Example 1 and in the basic technology description alone. The microcapsules produced have a particle size distribution in the range 1.00-3.00 mm.

Omega-3-fatty acids are produced into seamless microcapsules as described in Example 1 and the basic technology description. The microcapsules produced have a particle distribution in the range 1.00-3.00 mm.

The cyclosporine microcapsules and omega-3-fatty acid microcapsules are encapsulated into hard gelatin capsules using a Macofar Multi-Dosing Pellet Encapsulating Machine in the ratio 100:300 to give a Cyclosporine Active Content of 25 mg/capsule.

### EXAMPLE 9

Nimodipine is solubilised in a suitable solvent such as PEG 400 and formed into seamless microcapsules with an outer gelatin coating as hereinbefore described. These microcapsules have a size distribution of 3-6 mm.

The nimodipine microcapsules are then encapsulated into a hard gelatin capsule shell.

### EXAMPLE 10

Nimodipine seamless microcapsules are formulated as described in Example 9, however, the inherent release characteristics of the pellets are varied from Example 9, by increasing the wall thickness of the microcapsules. These microcapsules, when coated with the same polymer coating as in Example 1, provide a longer controlled release action.

### EXAMPLE 11

Cyclosporine is solubilised in a suitable medium chain triglyceride to which is added bile salt enhancer or bile salts, and formed into seamless microcapsules with an outer gelatin coating as herein before described. These microcapsules have a size distribution of 3-6 mm.

The cyclosporine and bile salt enhancer or bile salts microcapsules are then encapsulated into a hard gelatin capsule shell to give a cyclosporine content of 25-50 mg/capsule.

### EXAMPLE 12

Cyclosporine is solubilised in a suitable medium chain triglyceride, and formed into seamless microcapsules with an outer gelatin coating as herein before described. These microcapsules have a size distribution of 3-6 mm.

Bile salt enhancer or bile salts, are encapsulated into seamless microcapsules as described in Example 1. The cyclosporine microcapsules and bile salt enhancer or bile salt microcapsules are mixed and filled into hard gelatine capsules to give a cyclosporine content of 25-50 mg/capsule.

The cyclosporine and bile salt enhancer or bile salts microcapsules are then encapsulated into a hard gelatin capsule shell.

### EXAMPLE 13

Separate cyclosporine and bile salt enhancer or bile salt solutions are prepared as per Example 12.

The respective cyclosporine and bile salt enhancer or bile salt microcapsules are coated as in Example 1 to provide a different controlled release profiles. The bile salt enhancer or bile salts released prior to release of the cyclosporine.

### EXAMPLE 14

A P-glycoprotein inhibitor such as cyclosporine is solubilised in a suitable medium chain triglyceride, and formed into seamless microcapsules with an outer gelatin coating as herein before described. These microcapsules have a size distribution of 3-6 mm.

P-glycoprotein sensitive drugs such as bioflavonoids or antineoplastic molecules such as paclitaxel are encapsulated into seamless microcapsules as described in Example 1. The cyclosporine microcapsules and P-glycoprotein sensitive drug microcapsules are mixed and filled into hard gelatine capsules.
Ref:—Inhibition of P-glycoprotein by flavonoid derivatives in adriamycin-resistant human myelogenour leukaemia (K562/ADM) cells, Cancer Lett. 2002 Mar. 8; 177(1):89-93

### EXAMPLE 15

A blood brain barrier permeability modulators such as cyclosporine is solubilised in a suitable medium chain triglyceride, and formed into seamless microcapsules with an outer gelatin coating as herein before described. These microcapsules have a size distribution of 3-6 mm.

Drugs with limited blood brain barrier permeability such as Nimodipine are encapsulated into seamless microcapsules as described in Example 1.

The blood brain barrier permeability modulator microcapsules and limited blood brain barrier permeability compound microcapsules are mixed and filled into hard gelatine capsules.
Ref:—P-glycoprotein restricted transport of Nimodipine across blood brain barrier, Acta Pharmacol Sin. 2003 Sep., 24(9);903-6

### EXAMPLE 16

An intestinal permeability modulator such as nifidipine is formulated as in Example 1 and formed into seamless microcapsules with an outer gelatin coating as herein before described. These microcapsules have a size distribution of 3-6 mm.

A drug with limited intestinal permeability such as cyclosporine is solubilised in a suitable medium chain triglyceride, and formed into seamless microcapsules with an outer gelatin coating as herein before described These microcapsules have a size distribution of 3-6 mm.

The intestinal permeability modulator microcapsules and compound with limited intestinal permeability microcapsules are mixed and filled into hard gelatine capsules.
Ref: Nifidipine improves immediate 6 and 12 month graft function in cyclosporine(CyA) treated renal allograft recipients, Transpl Int. 1992; 5 Suppl 1; 869-92

### EXAMPLE 17

A P-glycoprotein inhibitor such as cyclosporine is solubilised in a suitable medium chain triglyceride, blended with a suitable pH modulator such as sodium citrate, and formed into seamless microcapsules with an outer gelatin coating as herein before described These microcapsules have a size distribution of 3-6 mm.

A P-glycoprotein and pH-sensitive drug such as Berberine is encapsulated into seamless microcapsules as described in Example 1. The cyclosporine microcapsules and P-glycoprotein-sensitive drug microcapsules are mixed and filled into hard gelatine capsules.
Ref:—Transport and uptake characteristics of a new derivative of berberine (CPU-86017 in human intestinal epithelial cell lnee: Caco-2, Acta Pharmacol Sin. 2003 Dec., 24(12):1185-91

### EXAMPLE 18

A hydroxymethyl-glutaryl-coenzyme (HMG-CoA) reductase inhibitor such as simvastatin is encapsulated into seamless microcapsules as described in Example 1.

A RES stimulator such as complex carbohydrate is solubilised and formed into seamless microcapsules with an outer gelatin coating as herein before described. These microcapsules have a size distribution of 3-6 mm.

The hydroxymethyl-glutaryl-coenzyme (HMG-CoA) reductase inhibitor microcapsules and RES stimulator microcapsules are mixed and filled into hard gelatine capsules.

### EXAMPLE 19

| | |
|---|---|
| Cyclosporine USP | 100 |
| Labrifil M1944 | 50-300 |
| Cremophor RH40 | 50-400 |
| Tween 80 | 0-200 |
| Polyethylene Glycol (grade 200; 300; 400; 600) | 50-200 |
| Ethanol | — |

Place labrifil cremophor and tweed (if required) in a suitable container and mix using a mechanical mixer until uniformly dispersed or dissolved (Label as Mix 1).

Place polyethylene glycol and ethanol (as required) in a suitable container. Add cyclosporine and mix using a mechanical mixer until the cyclosporine is completely solubilised. (Label as Mix 2).

Add Mix 1 to Mix 2 and continue mixing until all ingredients are uniformly dispersed. This is the Active Core Solution.

The active core solution is processed in the same way as described in Example 1 to form seamless microcapsules having a particle size range of 1.50-2.00 mm.

The cyclosporin microcapsules are then encapsulated into hard gelatin capsules.

### EXAMPLE 20

The microcapsules of Example 19 were administered to 8 healthy male volunteers and the bioavailability was compared with a conventional soft gel formulation of cyclosporin which is available under the Trade Mark Sandimmune from Novartis. The resulting bioavailability data demonstrate that the Tmax was reduced from 3.25 hr to 1.75 hr (100% increased speed to uptake) and the Cmax was increased from 580 ng/ml to 750 ng/ml (>40% increased drug uptake) when compared with the conventional formulation. Overall the AUC (area under the curve) was increased more than 20% when compared with the reference formulation. The data is presented in the following table. The bioavailability data are illustrated in the figure in which the continuous line represents bioavailability of the product of Example 19 and the interrupted line is bioavailability of the conventional reference product.

| | | Formulation of | | Sum- |
|---|---|---|---|---|
| PK Parameters | | example 19 | Reference | mary |
| Relative | Based | 103.14 ± 44.62 | | — |
| Bioavail- | on | 43.3 | | — |
| ability | AUCinf | | | |
| (%) | | | | |

| | | | |
|---|---|---|---|
| AUCinf | 3040.00 ± 1091.71 | 2586.23 ± 341.15 | >20% |
| (ng/mL · h) | | | Increase |
| CV % | 35.9 | 13.2 | |
| AUClast | 2855.09 ± 1043.08 | 2410.68 ± 332.93 | >20% |
| (ng/mL · h) | | | Increase |
| CV % | 36.5 | 13.8 | |
| Cmax | 732.31 ± 273.67 | 581.06 ± 109.13 | ˜40% |
| (ng/mL) | | | Increase |
| CV % | 37.4 | 18.8 | |
| Tmax(h) | 2.00 ± 0.93 | 3.00 ± 1.31 | 100% |
| CV % | 46.3 | 43.6 | Faster |
| Median | 1.75 | 3.25 | |
| Range | (1.00-3.50) | (1.00-5.00) | |
| Thalf | 8.12 ± 0.50 | 7.95 ± 1.30 | Equiv- |
| (h) | | | alent |
| CV % | 6.1 | 16.3 | |

[image]

The present invention allows for an active pharmaceutical ingredient to be maintained in a liquid phase and then encapsulated into microcapsules. The present invention also allows the active ingredient to be maintained in its liquid phase thus enhancing the solubility of an insoluble partially soluble pharmaceutical ingredient.

This invention is not limited to the embodiments hereinbefore described which may be varied in detail.

1-61. (canceled)

62. A controlled release formulation in solid unit dosage form, said formulation comprising a multiplicity of seamless microcapsules, each microcapsule comprising one or more active ingredient in a liquid phase and having a predetermined release rate of each said active ingredient in the gastrointestinal tract following administration, the microcapsules collectively having one or more rates of release of each said active ingredient dependent on a predetermined permeability of the respective microcapsules.

63. The formulation according to claim 62, wherein the individual microcapsules have an average diameter in the range of from 100 to 10,000 microns.

64. The formulation according to claim 62, wherein the individual microcapsules have an average diameter in the range of from 250 to 8,000 microns.

65. The formulation according to claim 62, wherein the individual microcapsules have an average diameter in the range of from 500 to 5,000 microns.

66. The formulation according to claim 62, wherein the walls of the microcapsules have a thickness in the range of from 30 to 1,000 microns.

67. The formulation according to claim 66 wherein the walls of the microcapsules have a thickness in the range of from 100 to 500 microns.

68. The formulation according to claim 62, wherein the wall of each microcapsule is formed of a pharmaceutically acceptable, film-forming polymer or mixture of polymers which is soluble in the gastrointestinal tract.

69. The formulation according to claim 68, wherein the wall of the microcapsule is formed of a gel material.

70. The formulation as claimed in claim 69 wherein the gel material is gelatine.

71. The formulation as claimed in claim 70 wherein the gelatine is a bovine or porcine gelatine material.

72. The formulation as claimed in claim 69 wherein the gel material is selected from one or more of a starch, a high molecular weight polyethylene glycol, and agar.

73. The formulation according to claim 62, which is in the form of a blend of microcapsules having walls of variable, but predetermined, permeability so as to achieve immediate, intermediate or sustained release of active ingredient over a given time period in the gastrointestinal tract.

74. The formulation according to claim 62, wherein the microcapsules contain two or more active ingredients having different solubilities in the aqueous environment of the gastrointestinal tract.

75. The formulation according to claim 74, wherein the solubility of the or each drug is dependent on the pH of a given region of the gastrointestinal tract.

76. The formulation according to claim 62, wherein the microcapsules contain two or more active ingredients having different half lives following absorption from the gastrointestinal tract.

77. The formulation according to claim 62, wherein the walls of the microcapsules have inherent mucoadhesive properties and bind to the wall of the gastrointestinal tract during release of active ingredient therefrom.

78. The formulation according to claim 62, wherein the walls of the microcapsules have inherent enteric coating properties.

79. The formulation according to claim 62, wherein some or all of the microcapsules in the formulation have an enteric coating.

80. The formulation as claimed in claim 62 wherein the microcapsules contain an active ingredient suspended or solubilised in a solution of a permeability enhancer.

81. The formulation as claimed in claim 80 wherein the ratio of enhancer to drug is from 0 drug: 100 enhancer to 100 drug: 0 enhancer.

82. The formulation as claimed in claim 62 comprising microcapsules containing enhancer solution alone.

83. The formulation as claimed in claim 82 wherein the microcapsules containing the enhancer have a rate of release of enhancer to maximise the permeability enhancement of unabsorbed drug.

84. The formulation according to claim 62, wherein the liquid phase is an oil.

85. The formulation according to claim 74, wherein the or each active ingredient is dissolved in a polyol in the core of the microcapsules.

86. The formulation according to claim 85, wherein the polyol is a cellulose derivative.

87. The formulation according to claim 62, wherein the core contains one or more auxiliary agents selected from a pH controlling agent, an anti-oxidant, a humectant, a surfactant and a vasodilator.

88. The formulation according to claim 62, wherein the microcapsules contain up to 90% by weight of an active ingredient.

89. The formulation according to claim 62, wherein the microcapsules contain between 25 and 75% by weight of an active ingredient.

90. The formulation according to claim 62, wherein each formulation contains between 10 and 300 microcapsules.

91. The formulation as claimed in claim 62 wherein each formulation contains between 100 and 250 microcapsules.

92. The formulation according to claim 62, wherein the microcapsules are contained in a hard gelatine capsule.

93. The formulation according to claim 62, wherein the microcapsules are contained in a sachet which may be used as a sprinkle.

94. The formulation according to claim 62, wherein the walls of the microcapsules comprise more than one coating.

95. The formulation as claimed in claim 94 wherein the inner coat comprises a mucoadhesive which is coated with an outer protective enteric coat.

96. The formulation as claimed in claim 95 wherein the mucoadhesive is altered to ensure binding to specific areas of the gastrointestinal tract.

97. The formulation as claimed in claim 62 wherein the active ingredient is substantially insoluble.

98. The formulation as claimed in claim 97 wherein the active ingredient is nifedipine.

99. The formulation as claimed in claim 62 wherein the active ingredient is a lipid soluble ingredient.

100. The formulation as claimed in claim 99 wherein the active ingredient is gemfibrizol.

101. The formulation as claimed in claim 62 wherein the active ingredient is pH sensitive.

102. The formulation as claimed in claim 101 wherein the active ingredient is captopril.

103. The formulation as claimed in claim 101 wherein the active ingredient is sensitive to the pH environment in the stomach.

104. The formulation as claimed in claim 103 wherein the active ingredient is a proton pump inhibitor.

105. The formulation as claimed in claim 103 wherein the active ingredient is omeprazole.

106. The formulation as claimed in claim 62 wherein the active ingredient has low oral bioavailability.

107. The formation as claimed in claim 106 wherein the active ingredient is terfenedine.

108. The formulation as claimed in claim 62 wherein the active ingredient is poorly absorbed from or destroyed in the gastrointestinal tract.

109. The formulation as claimed in claim 108 wherein the active ingredient is selected from one or more of captopril, cyclosporin, calcitonin, heparins and heparinoids.

110. The formulation as claimed in claim 62 wherein the active ingredient is a poorly water soluble drug.

111. The formulation as claimed in claim 62 wherein the active ingredient is selected from one or more of a cardiovascular, an anti-diabetic agent, an anti-epileptic, an anti-infective, an anti-fungal agent, an anti-viral agent, an antipsychotic agent, an immunosuppressant, a protease inhibitor and a cyclic peptide.

112. The formulation as claimed in claim 62 wherein the active ingredient is selected from one or more of a peptide, a protein, a vaccine and an oligonucleotide, including covalent or non-covalent derivatives thereof.

113. The formulation as claimed in claim 62 containing an ACE inhibitor, an AT₁ blocker and a diuretic.

114. The formulation as claimed in claim 113 wherein the ACE inhibitor is selected from one or more of captopril and perindopril.

115. The formulation as claimed in claim 113 wherein AT₁ blocker is selected from one or more of losartan and valsartan.

116. The formulation as claimed in claim 113 wherein the diuretic is selected from any one or more of indapamide and hydrochlorothiazide.

117. The formulation as claimed in claim 62 containing a calcium channel blocker, an ACE inhibitor, a peripheral x-adrenergic blocker and a hydroxymethyl-glutaryl-coenzyme (HMG-CₒA) reductase inhibitor.

118. The formulation as claimed in claim 117 wherein the calcium channel blocker is selected from one or more of amlodipine and nimodipine.

119. The formulation as claimed in claim 117 wherein the ACE inhibitor is selected from one or more of lisinopril and ramipril.

120. The formulation as claimed in claim 117 wherein the periphenol x-adrenergic blocker is doxazosin.

121. The formulation as claimed in claim 117 wherein the HMG-CₒA reductive inhibitor is a statin.

122. The formulation as claimed in claim 121 wherein the statin is selected from one or more of pravastatin and simvastatin.
